(19) United States
(12) Patent Application Publication
(10) Pub. No.: US 2011/0263722 A1
(43) Pub. Date: Oct. 27, 2011

(54) Well Defined stars with segmented arms

(76) Inventors: Wojciech Jakubowski, Pittsburgh, PA (US); Patrick McCarthy, Pittsburgh, PA (US); Nicolay Tsarevsky, Dallas, TX (US); James Spanswick, Pittsburgh, PA (US)

(21) Appl. No.: 12/799,412

(22) Filed: Apr. 23, 2010

(51) Int. Cl. C08F 20/06; A61K 47/32

(52) U.S. Cl. 514/772.6; 526/214

(57) ABSTRACT

The present invention relates to the preparation and use of well defined star macromolecules wherein the composition of the arms are selected to induce self assembly when the multi-arm segmented star macromolecules are dispersed in a liquid. The self assembled star macromolecules modify the rheology of the dispersing media. When targeting use in aqueous based systems the inner shell of the star macromolecules comprise non-ionizable monomer units. Compositions comprising the self assemblable star macromolecules are suitable for use as rheology modifiers in a number of applications including cosmetic and personal care compositions.

### TECHNICAL FIELD OF THE INVENTION

The present invention relates to the preparation and use of well defined star macromolecules wherein the composition of the arms selected to induce self assembly when the multi-arm segmented star macromolecules are dispersed in a liquid. Compositions comprising the self assemblable star macromolecules are suitable for use as rheology modifiers in a number of applications including cosmetic and personal care compositions.

### BACKGROUND AND PRIOR ART

### Rheology Modifiers

This invention generally relates to well defined star macromolecules that self assemble in solution to provide a certain level of control over viscosity and consistency factors in many aqueous or oil based systems where control over the rheology is a concern. Applications include; water- and solvent-based coating compositions, paints, inks, antifoaming agents, antifreeze substances, corrosion inhibitors, detergents, oil-well drilling-fluid rheology modifiers, additives to improve water flooding during enhanced oil recovery, dental impression materials, cosmetic and personal care applications including hair styling, hair conditioners, shampoos, bath preparations, cosmetic creams, gels, lotions, ointments, deodorants, powders, skin cleansers, skin conditioners, skin emollients, skin moisturizers, skin wipes, sunscreens, shaving preparations, and fabric softeners, with the rheology modifier providing characteristics of high gel strength, highly shear thinning, forms versatile low viscosity soluble concentrations, and synergistic interactions with added agents to adjust their rheology profile to optimize properties such as sedimentation, flow and leveling, sagging, spattering, etc.

Therefore while utility of the disclosed star macromolecules will initially be exemplified in cosmetic applications their advantages are not limited to cosmetic and personal care compositions. The composition of each segment of the star thickening agents disclosed herein can be modified to provide high performance and value in applications such as those discussed above. Cosmetic and personal care compositions such as hair styling sprays, mousses, gels and shampoos, frequently contain resins, gums and adhesive polymers to provide a variety of benefits, for example, film-forming ability, thickening, sensory properties and hair shaping and setting. Polymers designed for use in such compositions generally focus on linear or graft copolymers which contain various monomers in an alternating, random or block configuration. The disclosed self assembling star molecules can provide similar or improved properties at lower concentrations; less than 10 wt % preferably less than 5 wt % and more preferably less than 1 wt % of the final product.

The thickeners used in cosmetic and body care preparations have to meet stringent requirements. First and foremost, they have to show high compatibility with other agents in the formulations and also if possible biodegradability so that many substances have to be ruled out from the outset for use in cosmetics. In addition, they should be universally useable in aqueous, emulsoidal, alcoholic and oil-containing bases, be readily incorporated into the formulations and lead to a rheology which enables the product to be easily, applied so that the final preparations can be removed from containers and distributed under clean and simple conditions.

Thickeners that are macromolecularly designed to provide the desired properties would be expected to be compatible with many other auxiliaries present in the formulations, more particularly with salts and surfactants. The thickener itself and the other auxiliaries should also lend themselves to ready incorporation into the formulation. The thickened preparations are also expected to show stable rheology and an unchanging physical and chemical quality even in the event of long-term storage and changes in pH and temperature.

Finally, the thickeners should be inexpensive to produce without causing significant environmental pollution.

In view of this complex requirement profile, it is clear why, even today, there is still a demand for new thickeners in the cosmetics field.

Accordingly, one problem addressed by the present invention was to provide cosmetic formulations which, after addition of only small quantities of a thickener, would be easy to apply and would leave the skin with a pleasant feeling. The formulations would be easy to distribute on the skin and in the hair without leaving a feeling of stickiness behind. They would have improved physical and chemical stability and would be highly compatible with the skin and scalp. In addition, the viscosity and consistency factors would be unaffected by additions of ions and other auxiliaries or by changes in pH and temperature.

Other agents that are included in the term “auxiliaries” include; surfactants, oils, fats and waxes, emulsifiers, silicone compounds, UV protectors, antioxidants, various water soluble substances, biogenic agents, deodorants, odor absorbers, antiperspirants, and germ and enzyme inhibitors. Such agents are disclosed in U.S. Pat. No. 6,663,855 and U.S. Pat. No. 7,318,929 herein incorporated by reference to provide definitions for those terms.

### Controlled Radical Polymerization Procedures

Controlled radical polymerization (CRP) has emerged during the past decade as one of the most robust and powerful techniques for polymer synthesis, as it combines some of the desirable attributes of conventional free radical polymerization (e.g., the ability to polymerize a wide range of monomers, tolerance of various functionality in monomer and solvent, compatibility with simple industrially viable reaction conditions) with the advantages of living ionic polymerization techniques (e.g., preparation of low polydispersity index (PDI=M_{w}/Mₙ) polymer and chain-end functionalized homo- and block (co)polymers). The basic concept behind the various CRP procedures is the reversible activation of a dormant species to form the propagating radical. A dynamic and rapid equilibrium between the dormant and the active species minimizes the probability of bimolecular radical termination reactions and provides an equal opportunity for propagation to all polymer (or dormant) chains.

Based on the number of publications in the past fifteen years here are three broadly applied CRP procedures.

Scheme 1 shows how they can be classified based on the mechanism of reversible activation: (a) stable free radical polymerization (SFRP, Scheme 1a), (b) degenerative chain transfer polymerization (DT, Scheme 1b), and (c) atom transfer radical polymerization (ATRP, Scheme 1c).

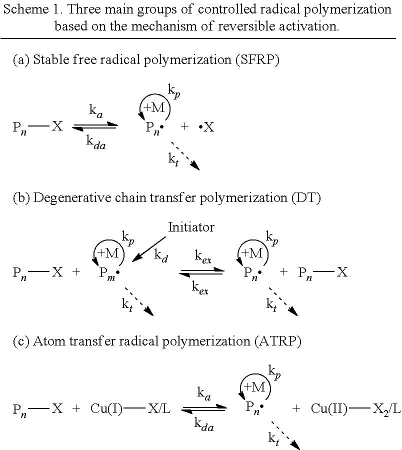

As shown in Scheme 1 various capping agents, “X”, are used for the different CRP procedures and they are summarized below in Scheme 2. They include stable nitroxides (Scheme 2a), transition metal complexes (Scheme 2b), halides with transition metal catalysts (Scheme 2c), iodine with catalysts (Scheme 2d), sulfur compounds (Scheme 2e), iodine (Scheme 2f), and organometal compounds (Scheme 2g).

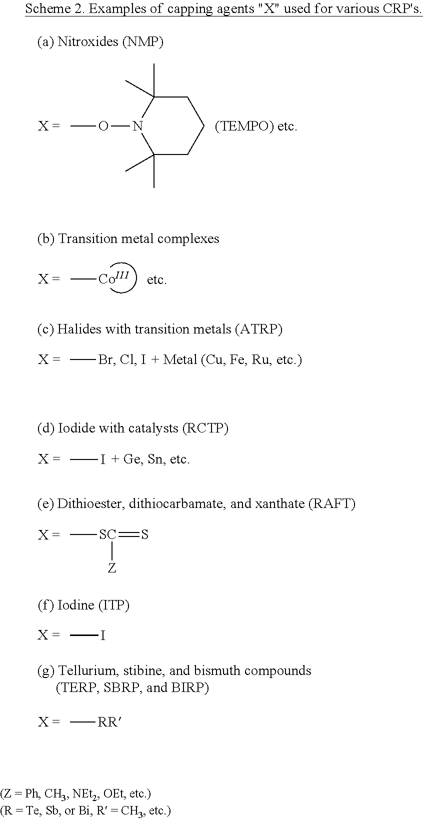

In the following discussion, and in the examples, we will use ATRP as an exemplary controlled radical polymerization process but the strategy disclosed for the preparation of star macromolecules with segmented arms and no remaining transfer/capping agent can be applied to any of the above polymerization processes. This ability to use different CRP's to form specific arm segments in the star macromolecule allows one to increase the range of monomers that can be incorporated into the star arms. For example a degenerative transfer process, including procedures “e”, “f” or “g”, can be used if one wishes to polymerize vinyl acetate from a multi-functional low molecular weight initiator which after hydrolysis forms stars with a polyvinyl alcohol inner shell, when segmented arms are formed.

The techniques, summarized in scheme 2, make it possible to overcome the limitations inherent in conventional radical polymerization, that is to say make it possible to control the length of the polymer chains while retaining control over the functionality at the terminus of each growing polymer segment as it is formed and therefore to obtain block copolymer structures. The controlled radical polymerization procedures make it possible to reduce the number of reactions in which the growing radical species is irreversibly deactivated, in particular the termination reactions, which in conventional radical polymerization, interrupt the growth of the polymer chain in an irreversible and uncontrolled way. In order to decrease the probability of termination reactions, provision has been made to block, in a temporary and reversible way, the growing radical species by forming so-called “dormant” activatable species with the aid of a bond of low dissociation energy.

Mention has been made of the possibility of using bonds of C-halide type (in the presence of metal/ligand complex). This is the procedure that has been described as atom transfer radical polymerization, also known under the abbreviation ATRP, see scheme 1c. This type of polymerization is reflected in control of the mass of the polymers which are formed and in a low polydispersity index for the chains. This process is illustrated in particular in Application WO 97/18247, the teaching of which can be drawn upon by a person skilled in the art in preparing the polymers coming within the scope of the present invention. The nature and the amount of the monomers, initiator(s), compound(s) comprising the transition metal and ligand(s) will be chosen by a person skilled in the art on the basis of his overall knowledge, according to the result desired.

In particular, the monomers “M” can be chosen, alone or as a mixture, from radically (co)polymerizable monomers comprising ethylenic unsaturation corresponding to the formula:

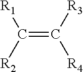

in which R₁, R₂, R₃ and R₄ are defined in incorporated references including Application WO 97/18247.

For the purpose of this invention, the term “independent,” when used to describe the relationship of radicals, atoms, substituents, functional groups, etc., means that each of the radicals, atoms, substituents, functional groups, etc. may be the same or different from the other, or some radicals, atoms, substituents, functional groups, etc., may be the same while the others may be different.

In an initial non-limiting exemplifying case the procedure employed for the preparation of the arms is known as initiators for continuous activator regeneration (ICAR) atom transfer radical polymerization (ATRP) or a related procedure names activators regenerated by electron transfer (ARGET) as discussed by two of the current inventors. [Matyjaszewski, K.; Jakubowski, W.; Min, K.; Tang, W.; Huang, J.; Braunecker, W. A.; Tsarevsky, N. V. Proceedings of the National Academy of Sciences of the United States of America 2006, 103, 15309-153141

In one exemplary controlled radical procedure, the ICAR ATRP approach, a low concentration of catalyst complex is employed to provide controlled growth of each arm from a well defined multifunctional core initiator and the excess higher oxidation state transition metal complex formed by a low incidence of radical/radical termination reactions is reduced to the activator state by reaction with radicals formed by controlled degradation of an added standard free radical initiator. In an ICAR ATRP the concentration of the initially added transition metal complex can be less than 500 ppm, preferably less than 100 ppm.

Without being limited by the following explanation it is believed that in an ICAR ATRP the rate of the reaction is predominately derived from the rate of decomposition of the added standard free radical initiator while controlled growth of each arm of the star is dependent on the selected transition metal complex.

### Star Macromolecules

Star polymers are nano-scale materials with a globular shape. They can optionally possess multiple segmented arms and a high-density of peripheral functionality. The spherical shape and dense structure of this type of polymer are expected to provide a suite of properties and functions different from that of linear polymers. Indeed the preparation of functionalized star polymers with uniformed size and multiple arms with site specific functionalities is presently the subject of extensive academic and industrial interest due to their unique structure and potential applications in cosmetics, drug delivery systems, coatings, membranes and lithography.

Synthesis of star polymers is most often accomplished by “living” polymerization techniques via one of three strategies:
“core-first” which is accomplished by growing arms from a multifunctional initiator;
“coupling-onto” involving attaching preformed arms onto a multifunctional core and the
“arm-first” method which involves cross-linking preformed linear arm precursors using a divinyl compound.

The “core-first” method is exemplified by the use of a multifunctional initiator in a living polymerization process most often employing living ionic polymerization systems. This approach is also called the “grafting from” approach where the arms of the star are grown from a preformed functionalized core molecule or particle, see U.S. Pat. Nos. 5,763,548 and 6,627,314 for examples of core first synthesis of star molecules using the ATRP procedure.

The “arm-first” strategy can be further sub-categorized according to the procedure employed for star formation. One method is chain extension of a linear arm precursor with a multivinyl cross-linking agent, and the other is coupling linear polymer chains to a multifunctional linking agent, or “grafting-onto” a multifunctional core. The development of living/controlled radical polymerization has revitalized the field of star polymer synthesis, especially for functional star polymers and various star polymers with many arms have been synthesized, mostly using these two “arm-first” methods. [U.S. Pat. Nos. 6,512,060 and 6,627,314]

Indeed the “arm-first” approach to star synthesis has been the subject of all living polymerizations systems. Anionic polymerization was described by Rempp [Zilliox, J. G., P. Rempp, et al. (1968). “Preparation of star-shaped macromolecules by anionic copolymerization.” Journal of Polymer Science. Polymer Symposia No. 22 (Pt. 1): 145-56], and cationic has been summarized by Kanaoka. [Kanaoka, S., N. Hayase, et al. (2000). “Synthesis of star-shaped poly(vinyl ether)s by living cationic polymerization: pathway for formation of star-shaped polymers via polymer linking reactions.” Polymer Bulletin (Berlin) 44(5-6): 485-492; Shibata, T., S. Kanaoka, et al. (2006). “Quantitative Synthesis of Star-Shaped Poly(vinyl ether)s with a Narrow Molecular Weight Distribution by Living Cationic Polymerization.” Journal of the American Chemical Society 128(23): 7497-7504.] However these living ionic polymerization procedures cannot be used to polymerize polar or neucleophylic monomers.

While all above controlled polymerization procedures are suitable for preparation of the disclosed self assembling star macromolecules the present disclosure will exemplify the preparation of the self assembling multi-arm stars using the “core first” strategy and ATRP.

The Controlled Radical Polymerization process (CRP) known as ATRP; disclosed in U.S. Pat. Nos. 5,763,546; 5,807,937; 5,789,487; 5,945,491; 6,111,022; 6,121,371; 6,124,411: 6,162,882: 6,407,187; 6,512,060; 6,538,091; 6,541,580; 6,759,491; and 7,332,550; and in U.S. patent application Ser. Nos. 10/887,029; and 11/990,836 and discussed in numerous publications listed elsewhere with Matyjaszewski as co-author, which are hereby incorporated into this application. The disclosed ATRP procedures describe the preparation of polymers displaying control over the polymer molecular weight, molecular weight distribution, composition, architecture, functionality and the preparation of molecular composites and tethered polymeric structures comprising radically (co)polymerizable monomers, and the preparation of composite macromolecular structures under mild reaction conditions.

A homogeneous ATRP reaction was first disclosed in WO 97/18247 which is hereby incorporated by reference to provide an indication of the scope of the procedure and the range of suitable hydrophilic and hydrophobic (co)polymerizable monomers that can be used in the disclosed procedure. ATRP generally requires an alkyl halide, or pseudo-halide, as an initiator (R—X) and a transition metal complex (e.g., Cu, Ru, Os, Mo, Fe, etc.) as a catalyst. ATRP involves homolytic cleavage of an R—X bond by a partially soluble (WO 96/30421) transition metal complex in a lower oxidation state, such as Cu(I)-X/L (with a rate constant kₐ), followed by propagation (with a rate constant kₚ) and reversible deactivation of the propagating chain radical (R^{}) (with a rate constant k_{da}) by the higher oxidation state catalyst complex, Cu(II)-X₂/L where L is a ligand that solubilizes the transition metal and adjusts the activity of the formed catalyst complex. The reaction progresses by repetitive transfer of halogen, or pseudo-halogen atoms, to and from the transition metal complex, as shown in Scheme 1c.

The development of new catalysts and procedures for the reduction of the concentration of the catalyst are topics of great interest for industrial acceptance of ATRP. For example, the development of two new initiation techniques, known as activators regenerated by electron transfer (ARGET) and initiators for continuous activator regeneration (ICAR) ATRP, permitted polymerizations with dramatically lower catalyst concentrations, which, for many applications do not require removal of the catalyst from the final product. These procedures are disclosed in applications WO 2005/087819 and WO 2007/025310 whose text is also included by reference.

Recent studies on bond dissociation energy (BDE) of alkyl (pseudo)halides proved that the activity of ATRP initiators depends reciprocally on the alkyl (pseudo)halide BDE, [Lin, C. Y.; Coote, M. L.; Gennaro, A.; Matyjaszewski, K. J. Am. Chem. Soc. 2008, 130, 12762-12774]. A systematic study on the effects of the initiator structure on initiation activity was recently reported, [Tang, W.; et al.; J. Am. Chem. Soc. 2008, 130, 10702-10713].

Indeed, since ATRP is such a useful procedure and can incorporate polymer segments prepared by other polymerization procedures it has been employed for the preparation of linear polysiloxane block copolymers for use in topical cosmetic and personal care compositions as disclosed in U.S. Pat. No. 6,365,672. ATRP has also been employed for synthesis of graft polymers, comprising hydrophobic and hydrophilic segments, for cosmetic applications U.S. Pat. No. 5,986,015. In the '015 patent the polymeric backbone has a weight average molecular weight of from about 500 grams/mole to about 200,000 grams/mole, wherein the polymeric backbone and the plurality of polymeric side chains form hydrophilic and hydrophobic graft polymers having a weight average molecular weight of from about 16,000 grams/mole to about 10,000,000 grams/mole.

There is a series of US patents discussing the use of star polymers for application in the field of make-up; U.S. Pat. Nos. 6,476,124; 6,552,146; 6,692,733; 6,723,789; 6,737,071; and 6,946,525. The formed materials are capable of being applied to the skin, semi-mucous membranes and/or mucous membranes. These star macromolecules are prepared by conducting an ATRP from a low molecular weight core molecule with a low specific number of initiating sites preferably between 4 and 10, as first disclosed in WO 96/30421. However the star copolymers prepared by these prior art disclosed core first ATRP procedures retain the radically transferable atom, most often a halogen, at the periphery of the star molecule. This is undesirable for an environmentally acceptable “natural” product.

We disclose herein a process for the preparation of star macromolecules that overcomes the prior art limitations.

### DESCRIPTION OF INVENTION

It is to be understood that this invention is not limited to specific compositions, components or process steps disclosed herein, as such may vary. It is also to be understood that the terminology used herein is for the purpose of describing particular embodiments only, and is not intended to be limiting.

### BRIEF DESCRIPTION OF THE DRAWINGS

The features and advantages of the present invention may be better understood by reference to the accompanying figures, in which:

FIG. 1: Presents the kinetic data from ICAR ATRP of hydroxyethyl acrylate from a monofunctional initiator, Et₂BrMM.

FIG. 2: Evolution of molecular weights and polydispersity with monomer conversion in the ICAR ATRP of HEA (containing inhibitor) initiated by Et₂BrMM.

FIG. 3: Presents A) the kinetic data, and B) evolution of molecular weight and polydispersity with conversion, in the ICAR ATRP of hydroxyethyl acrylate in protic media conducted from multifunctional core initiators to form 3-arm; 4-arm and 6-arm stars using tri-, tetra-, and hexa-functional initiators.

FIG. 4: Viscosity measurements of 1.5 wt % of the star macromolecules in a shampoo.

As used in the specification and appended claims, the singular forms “a,” “and,” and “the” include plural referents unless the context clearly dictates otherwise. Thus, for example, reference to “a polymer” may include more than one polymer, reference to “a substituent” may include more than one substituent, reference to “a monomer” may include multiple monomers, and the like.

In describing and claiming the present invention, the following terminology will be used in accordance with the definitions set out below.

The term “polymer” is used to refer to a chemical compound that comprises linked monomers, and that may or may not be linear; the term “(co)polymer” includes homo polymers and copolymers. “Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

Unless otherwise indicated, all numbers expressing quantities of ingredients, time, temperatures, and so forth used in the present specification and claims are to be understood as being modified in all instances by the term “about.” Accordingly, unless indicated to the contrary, the numerical parameters set forth in the following specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, may inherently contain certain errors necessarily resulting from the standard deviation found in their respective testing measurements.

The terms “narrow molecular weight distribution” or “narrow polydispersity” are used herein to mean a molecular weight distribution or polydispersity of less than 2.0 preferably less than 1.5.

It is also to be understood that the terminology used herein is only for the purpose of describing the particular embodiments and is not intended to be limiting.

The term multi-arm star indicates that a star shaped macromolecule with three or more arms linked at the core of the star is formed.

The core of the star molecule can optionally comprise degradable functionality.

Well defined multiarm stars with segmented arms are the preferred topology for the present invention as they can adopt a globular shape wherein each arm can chain extend in a selected targeted solvent to attain a highly swollen structure or highly chain extended structure.

The present disclosed star macromolecules comprise inner shell segments that are stable in the presence of salts and over a range of pH, and in one embodiment do not contain control agents, in addition to being more efficient as they can be employed at lower concentration in the formulations.

The composition of the stars of the present invention can be selected so that one or more segments in the star macromolecules can interact with other components of a cosmetic product to provide the final physical properties desired for the final product.

The invention is not limited to the specific compositions, components or process steps disclosed herein as such may vary.

Citation or identification of any document in this application is not an admission that such document is available as prior art to the present invention.

In one embodiment of the disclosed star macromolecule the terminal segments on one or more of the tethered arms can comprise a polymer composition that is compatible with added agents, to provide an additional degree of self assembly thereby providing an additional level of control over thickening properties. This embodiment is exemplified below by incorporation of an aliphatic chain end to provide a thickening agent compatible with hydrophobic functional groups present in added commercially available surfactants.

Commercially available surfactants include anionic, nonionic, cationic, zwiterionic and amphoteric surfactants. Typical examples of anionic surfactants are soaps, alkyl benzenesulfonates, alkanesulfonates, olefin sulfonates, alkylether sulfonates, glycerol ether sulfonates, .alpha.-methyl ester sulfonates, sulfofatty acids, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethion-ates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside and Mineraloladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, .alpha.-olefin sulfonates, ether carboxylic acids, fatty acid glucamides, alkylamidobetaines, amphoacetals and/or protein fatty acid condensates, preferably based on wheat proteins. The composition of the shell can be selected to provide desired degree of interaction between the shell and surfactant(s).

It has been found that cosmetic preparations in which the disclosed star macromolecules are used as viscosity- and consistency-increasing factors show advantageous rheological behavior.

Even in small quantities, the star macromolecules used have an excellent thickening effect.

Even systems with low surfactant contents can be thickened.

The rheology of the formulations remains unchanged even after prolonged storage and despite changes in temperature.

The formulations are highly compatible with the skin and scalp. The small quantities of polymers lead to a pleasant, non-sticky feeling on the skin so that hair fibers are also prevented from sticking together.

The preparations show high physical and chemical stability, even when the composition contains high salt concentrations.

In the following discussion, some of the actual experiments are identified using the numbering system employed in the note books. There is no significance other than identification to the numbering system.

The present invention will be initially exemplified by the preparation of a multi-arm star macromolecule wherein the number of arms in the star macromolecule is between 3 and 25, preferably between 3 and 15, with segments selected to induce self assembly wherein the self assemblable star macromolecules are suitable for use as thickening agents or rheology modifiers in cosmetic and personal care compositions at low concentrations of the solid in the thickened solution, less than 5 wt %, preferably less than 2 wt %.

The structure of an exemplary new thickening agent, or rheology modifier, is a multiarm star copolymer wherein one or more segments in the arms of the star macromolecule are prepared by controlled radical polymerization (CRP) using a well defined multifunctional low molecular weight soluble molecule as initiator for the CRP. When directed towards use in aqueous systems the inner segments of the arms, i.e. those segments tethered close to the core and optionally the core, are hydrophilic, and other, outer shell segments are hydrophobic. When directed towards use in non-aqueous or oil based systems the phylicity of the inner and outer segments in the arms of the star can be reversed.

In a specific embodiment the inner segments of the arms comprise non-ionizable hydrophilic segments selected to make the star macromolecules compatible with solutions further comprising dissolved/dispersed salts thereby providing rheology modifiers that are additionally stable over a range of pH, from 1 to 14.

Scheme 3 shows a purely exemplary series of multifunctional initiators that were used to provide stars with 3, 4 or six arms in the following examples section. These specific multifunctional initiators were prepared by esterification of commercially available alcohols with an acid further comprising a radically transferable atom or group, in this instance bromine. Indeed many multifunctional structures can be incorporated into the core molecule for the star composition according to the invention. Esterification of any hydroxyl-molecule can be utilized to form a multifunctional initiator e.g. in one embodiment of the invention the multifunctional core of the star can comprise natural biodegradable polysaccharides.

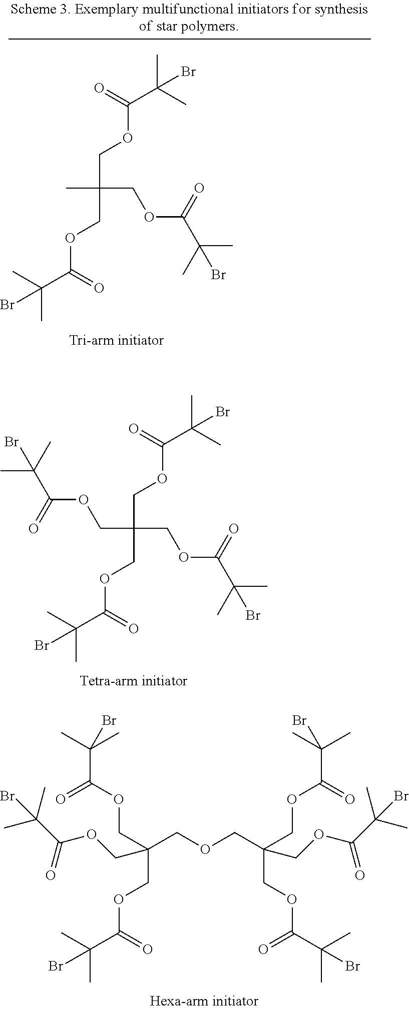

In Scheme 3 the initiator on the right, the hexa-arm initiator, can be considered an example of two initiators with the topology and composition of the initiator on the left, the tri-arm initiator, linked by an ether functionality. Indeed this could be considered as an exemplary precursor of a “dendritic” core structure. A simple procedure for the preparation of a “dendritic” core comprising initiating functionality by the controlled polymerization of AB* monomers was disclosed in WO1997/018247 and an improvement regarding appropriate selection of ligand and catalyst to increase reaction rate and modify the topology of the core in WO 1998/040415. Therefore in one embodiment of the invention the multifunctional initiator forming the core of the star, C in formula (1) is prepared using an AB* inimer.

Such AB* monomers can also be employed to increase the number of arms in a star macromolecule formed from a multi-functional initiator with a low number of initiating sites. When an AB* monomer is added to an ongoing polymerization of the first arms the unsaturated functionality is incorporated into the growing arm and the attached initiator functionality of the AB* monomer forms a three point branching point in the arm by initiating polymerization of mono-vinyl monomers present in the system and results in an increase in the number of tethered chains as the polymerization progresses.

The AB* monomer can comprise a degradable link between the unsaturated vinyl group and the initiator group.

Therefore in one embodiment of the invention for an ATRP “grafting from” reaction the core multifunctional initiator “C” comprises three or more initiating functionalities which are used to prepare a polymer or segmented copolymer, (S1)ₚ₁-(S2)ₚ₂, the “star” structure of which can be illustrated, in a general way, by the following formulae, (I) or (II):

C-[(S1)ₚ₁-(S2)ₚ₂]ₙ (I)

or,

{C}_{L}-[(S1)ₚ₁-(S2)ₚ₂]_{n′} (II)

in which:
C represents a polyfunctional future star centre or core, with a functionality of “n”, n being an integer greater than or equal to 3, preferably between 3 and 25, and {C}_{L}- represents the situation when several, two or more, (L) polyfunctional C centers are linked together to provide a central core initiator with an increased number of initiating moieties;
[(S1)ₚ₁-(S2)ₚ₂] represents a polymeric chain, also known as an “arm” of the star, composed of different polymerized monomers wherein segment S1 comprises an inner arm segment comprising one or more monomer units and S2 comprises a second, outer arm oligo-polymer segment on each branch, [(S1)ₚ₁-(S2)ₚ₂], being identical or different and being covalently grafted from the centre C;
n and n′ being the initial number of initiating functionalities on the polyfunctional center or core which is greater than or equal to 3, preferably of between 3 and 25;
p1 being the degree of polymerization of monomers comprising segment S1 which is greater than or equal to 2, preferably of between 10 and 20,000;
p2 being the degree of polymerization of monomers comprising segment S2 which is greater than or equal to 1, preferably of between 1 and 1,000.

The polymer chains are preferably provided in the form of blocks with a molecular mass of the arms are greater than or equal to 500 but which can range up to 2,000,000. However any specific segment in the copolymer can comprise a molar mass as low as 50.

The atom transfer radical trapping procedure discussed in detail below can also be employed using initiators of structure formula (1) where n is 3 or more to couple two or more initiator molecules to provide initiators of structure formula (2).

An approach to incorporate multiple initiating sites while also introducing degradability would be to functionalize natural products, such as polysaccharides, with initiator functionality using simple esterification reactions.

When targeting use in aqueous based systems the hydrophobic outer shell segments, —(S2)ₚ₂, in the above formulae can comprise one or more units wherein a segment or substituent is a “fatty phase” that can comprise conventional volatile or non-volatile oils, gums and/or waxes of animal, vegetable, mineral or synthetic origin, alone or as mixtures, in particular: linear, branched or cyclic, volatile or non-volatile, molecules and can include silicone oils which are optionally organomodified; gums which are liquid at room temperature; mineral oils, such as liquid paraffin and liquid petrolatum; oils of animal origin, such as perhydrosqualene or lanolin; oils of vegetable origin, such as liquid triglycerides, for example sunflower, maize, soybean, jojoba, gourd, grape seed, sesame, hazelnut, apricot, macadamia, avocado, sweet almond or castor oils, triglycerides of caprylic/capric acids, olive oil, groundnut oil, rapeseed oil or coconut oil; synthetic oils, such as purcellin oil, isoparaffins, fatty alcohols or esters of fatty acids; fluorinated and perfluorinated oils or fluorinated silicone oils; waxes chosen from known animal, fossil, vegetable, mineral or synthetic waxes, such as paraffin waxes, polyethylene waxes, carnauba or candelilla waxes, beeswaxes, lanolin wax, chinese insect waxes, rice wax, ouricury wax, esparto wax, cork fiber wax, sugarcane wax, japan wax, sumach wax, montan wax, microcrystalline waxes, ozokerite, the waxes obtained by the Fischer-Tropsch synthesis, silicone waxes or their mixtures.

In a preferred embodiment of the invention when the star macromolecule is designed to function as a thickening agent for an aqueous based system the inner shell or core of the star comprises water soluble non-ionizable monomer units. The term “monomer units” indicates that a first monomer has been polymerized and the resulting polymer comprises the polymerized monomer units distributed along the polymer backbone. Suitable radically copolymerizable monomers are listed in incorporated references and those particularly suited for the present star macromolecules targeting thickening agents stable in ionic systems include 2-hydroxylethyl (meth)acrylates, hydroxypropyl (meth)acrylates, glycidyl (meth)acrylates, PEO-oligo (meth)acrylates, (meth)acrylamides, allyl alcohol and vinyl pyrrolidone.

In one embodiment of the invention after the hydrophilic segment is grown from the multifunctional core initiator or macroinitiator molecule the radically transferable atom or group on the periphery of the first formed multi-armed star is converted into an oleophobic segment in a novel atom transfer radical trapping reaction.

A non-limiting explanation of the atom transfer radical trapping procedure that tethers a hydrophobic segment to each arm is provided in a simplified schematic in Scheme 4 wherein the capping agent of a CRP, in case of an ATRP the transferable atom, in the schematic a bromine atom, present on the ω-terminus of a linear polymer chain prepared by an ATRP procedure is activated by the transition metal complex in the presence of radicals formed by degradation of an agent selected to form radicals in the presence of an external stimulus such as light or heat.

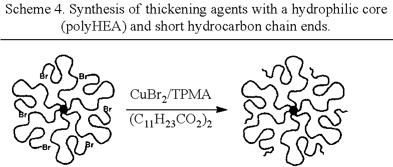

Lauryl peroxide is used as an exemplary agent in scheme 4. The radical formed by activating the dormant polymer chain end by the transition metal complex preferentially reacts with a lower molecular weight radical formed by decomposition of the added radical source to tether the radical to the ω-terminus of the polymer chain. The halogen chain ends of the first exemplary P(HEA) star polymers can be replaced in the presence of an excess of a radical source. An excess of low molecular weight radical source is employed to increase the fraction of cross coupling between the star-macro-radicals and low molecular weight radicals formed by decomposition of the radical source in the presence of small amount of a Cu(II) complex. The radical source provides radicals that both reduce the complex (similar to ICAR) and couple with the radicals generated via activation of the dormant polymer or arm chain ends by the generated Cu(I) activator.

This atom transfer radical trapping procedure can be considered a generally applicable procedure for removal of a halogen, or any radically transferable atom or group from a polymer chain end using continuous regeneration of the ATRP activator and cross-coupling. Furthermore it can be applied to any of the polymers prepared by any of the CRP procedures described in Scheme 2 when suitable procedures for chain end activation is conducted in the presence of an excess of low molecular weight radicals further comprising the desired oligo-polymer segment or to attach a suitable functional group that can be used in a second step to attach the selected segment.

In the present preferred procedure this process is conducted on the ω-terminus of each arm of the multifunctional star macromolecule formed by controlled polymerization of a hydrophilic arm from a multifunctional compact core molecule as illustrated in Scheme 4 and in greater detail in scheme 4, for the decomposition of lauryl peroxide. When a hexa-functional initiator is employed the formed star macromolecule with six segmented arms can be represented by the figure shown in Scheme 4. Such a star macromolecule can be used as a thickening agent and possesses the additional desired property of being insensitive to added salts is provided by the inner hydrophilic core of the star which additionally comprises non-ionizable monomer units.

In another embodiment the “fatty phase” can be incorporated by conducting a normal activation procedure in the presence of a saturated α-olefin that does not contain an activating substituent next to the olefin bond and once it has added to the chain end the dormant species cannot be reactivated. Such olefins with low molecular weight are commercially available. Higher molecular weight species can be prepared by telomerization of ethylene or ethylene propylene mixtures. [Kaneyoshi, H.; Inoue, Y.; Matyjaszewski, K. Macromolecules 2005, 38, 5425-5435.]

In another embodiment the “fatty phase” can be incorporated by conducting a normal activation procedure in the presence of a few monomer units comprising a substituent that comprises hydrophobic properties, such as lauryl (meth)acrylate or styrene based monomer units with a sufficiently long alkyl-group in the para-position. In such a first formed segmented copolymer the transferable control agent can be removed by the novel procedures disclosed herein or by employing the atom transfer radical addition or atom transfer coupling procedures disclosed in WO 98/040415.

The example detailed in scheme 4 shows formation of the lower molecular weight radical by decomposition of a peroxide, lauryl peroxide. A similar reaction can involve the decomposition of an azo compound such as AIBN. AIBN can be used to generate a radical (R^{}) that can be employed to attached a nitrile group to the terminus of each of the first formed star arms that can be subsequently employed in subsequent azide-alkyne “click” reactions to attach oligomers with complementary functionality to the first formed star after removal of the control agent [WO 2005087818] in a second functionalization reaction that tethers the desired outer shell segments, -(S2)ₚ₂ to the arms of the first formed star polymer.

In a non-limiting explanation of the mechanism of operation it is envisioned that the peripheral hydrophobic arm segments interact with hydrophobic segments in adjacent star molecules to self-assemble into a three dimensional array when dispersed in aqueous media or optionally interact with surfactant molecules that form micelles in the dispersion medium self-assemble into a shear sensitive three dimensional physically crosslinked network array.

In a non-limiting initial exemplary procedure detailed below in the examples the non-ionizable inner shell, (S1) was formed by a controlled radical polymerization of 2-hydroxyethyl acrylate.

The hydrophobic outer shell (S2) on the formed star can comprise oleophylic segments selected to interact with added surfactants to provide a self organized macromolecular gel thereby modifying the rheology of the solution.

Surfactants that can interact with the outer shell of the first segmented star macromolecule include anionic, nonionic, cationic zwitterionic and amphoteric surfactants.

In another embodiment of the invention the phobicity of the inner arm segments and the outer shell segments can be reversed providing a star macromolecule with a hydrophobic core and a hydrophilic shell that can be employed as a thickening agent for oil based formulations.

In another embodiment part way through the (co)polymerization of the monomer units that will comprise the inner shell of the star macromolecule a fraction of AB* monomer units equal to the number of initial initiating sites can be added to form a branch in the growing polymer chain thereby increasing the number of active chain ends for conversion to oleophilic units. This procedure increases the number of outer arm segments without increasing congestion at the core of the star.

### EXAMPLES AND DISCUSSION OF EXAMPLES

### Abbreviations

HEA hydroxyethyl acrylate
TPMA tris(2-pyridylmethyl)amine
Et₂BrMM diethylbromo methylmalonate
AIBN, azobisisobutyronitrile
EtOH ethanol

In one non-limiting example for the environmentally benign procedure for the preparation of a star shaped macromolecule designed to act as a rheology modifier an ICAR ATRP of HEA was studied in order to prepare well-defined star copolymers, which could be used for further functionalization reactions.

These are the first examples of ICAR ATRP carried out in protic media and a comparator example was conducted using a monofunctional initiator to determine conditions for the preparation of star molecules.

### Comparitor Example 1C

### Exemplary Synthesis of a Linear Polymer by Conducting an ICAR ATRP of HEA in Protic Media

The antioxidant present in HEA was removed and then 20 mL, (22.12 g, 0.190 mol), HEA and EtOH (20 mL) were added to a 100 mL round bottomed flask, and a magnetic stir bar was added to mix the reagents. Separately, a stock solution containing CuBr₂ (0.0088 g, 3.94×10⁻⁵ mol), TPMA (0.0115 g, 3.94×10⁻⁵ mol) and AIBN (0.0156 g, 9.50×10⁻⁵ mol) in DMF (10 mL) was prepared. The stock solution (5 mL, corresponding to 1.97×10⁻⁵ mol of CuBr₂ and TPMA, and 4.75×10⁻⁵ mol of AIBN) was then added to the mixture of monomer and solvent, followed by Et₂BrMM (182 μL, 9.525×10⁻⁴ mol, corresponding to DP_{n,targeted}=200. The flask was capped with a rubber septum and was then cooled in an ice bath (to minimize solvent evaporation). The liquid was purged with nitrogen for 1 h and the polymerization was then carried out at 65° C.

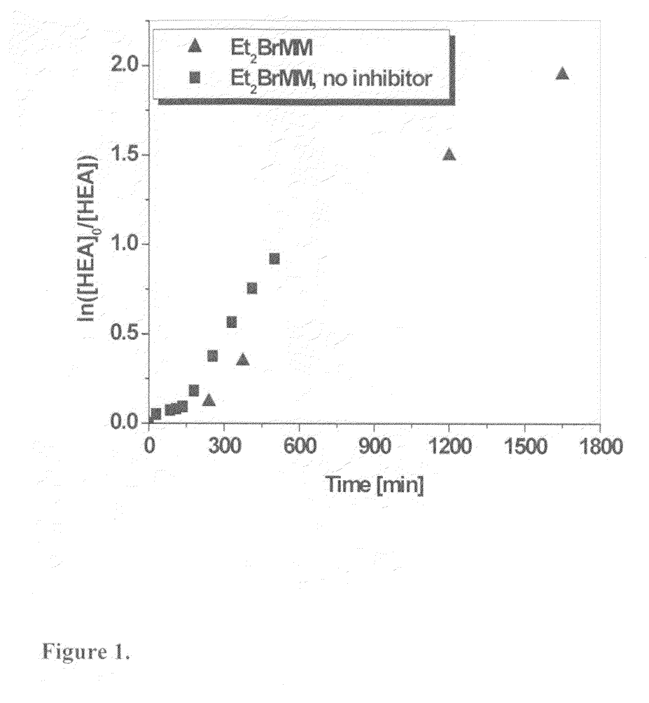

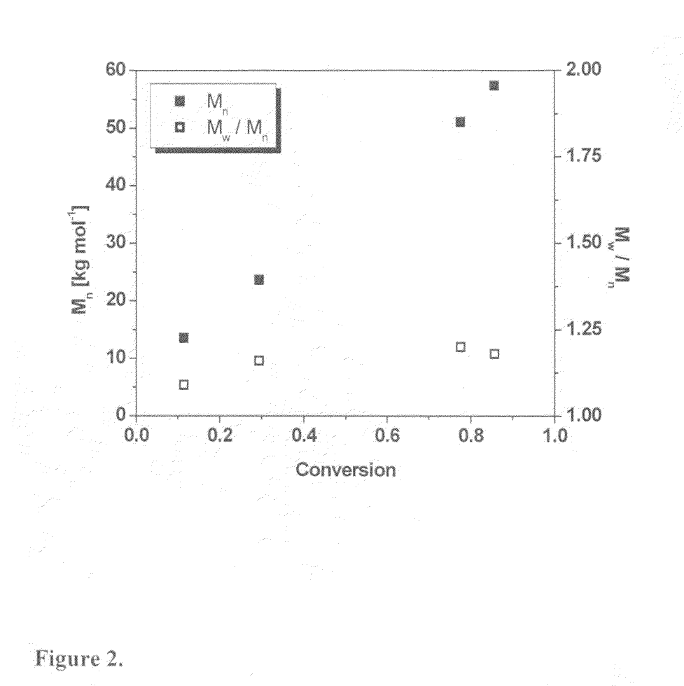

The kinetic plot is shown in FIG. 1. An induction period (ca. 2 h) was observed, which could possibly be due to the slow rate of addition of methylmalonate radical to HEA. When 2-bromoisobutyrate initiators were used in the polymerization (see below), the induction period was much shorter. The same reaction was carried out using HEA containing inhibitor, and 10 mL of EtOH. The reaction was somewhat slower and the induction period was slightly longer than in the first case (FIG. 1), although the degree of control over the polymerization was excellent (FIG. 2).

### Examples

### Synthesis of Star (Co)Polymers Synthesized by ICAR ATRP of HEA in Protic Media, Using the Initiators Shown in Scheme 3

### Example 1

### Synthesis of a Tri-Arm Star (nvt-08-007-51)

The reaction was carried out under conditions identical to the conditions described above for the linear comparator. HEA (20 mL, 22.12 g, 0.190 mol, of inhibitor free monomer) and EtOH (20 mL) were added to a 100 mL round bottomed flask and a magnetic stir bar was added to mix the reagents. Separately, a stock solution containing CuBr₂ (0.0088 g, 3.94×10⁻⁵ mol), TPMA (0.0115 g, 3.94×10⁻⁵ mol) and AIBN (0.0156 g, 9.50×10⁻⁵ mol) in DMF (10 mL) was prepared. The stock solution (5 mL, corresponding to 1.97×10⁻⁵ mol of CuBr₂ and TPMA, and 4.75×10⁻⁵ mol of AIBN) was then added to the mixture of monomer and solvent, followed by addition of the trifunctional ATRP initiator (0.5402 g, 9.525×10⁻⁴ mol). The flask was capped with a rubber septum and then cooled in an ice bath (to minimize solvent evaporation) while the liquid was purged with nitrogen for 1 h. The polymerization was then carried out at 65° C.

### Example 2

### Synthesis of a Four-Arm Star (nvt-08-007-42)

The reaction was carried out at conditions identical to the ones described above but the tetrafunctional 2-bromosiobutyrate initiator was used (0.6973 g, 9.525×10⁻⁴ mol). The initiator did not fully dissolve in the cold mixture of monomer and EtOH but did dissolve completely within 4-5 minutes after heating the mixture to 65° C.

### Example 3

### Synthesis of a Six-Arm Star (nvt-08-007-55)

The reaction was carried out at conditions identical to the ones described above but the hexafunctional 2-bromosiobutyrate initiator was used (1.0937 g, 9.525×10⁻⁴ mol). The initiator did not fully dissolve in the mixture of monomer and EtOH but dissolved completely within 2-3 minutes after heating the mixture to 65° C.

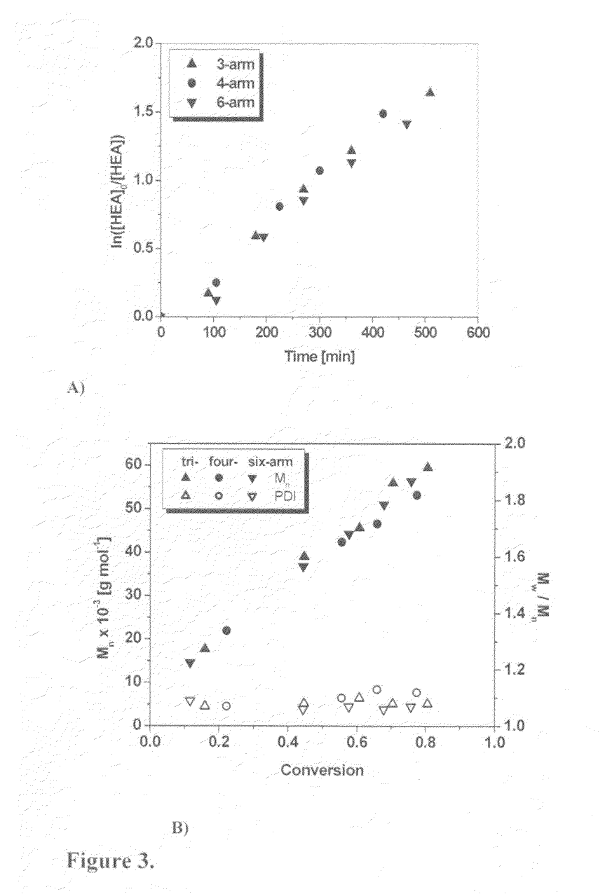

The kinetic plots and the evolution of molecular weights and polydispersity with conversion for the synthesis of various stars are presented in FIG. 3 which confirms that excellent control over the polymerization was achieved.

### Example 4

### Synthesis of Six-Arm Star of Higher Molecular Weight (nvt-08-007-56)

The reaction was carried out under conditions identical to those described above but the hexafunctional 2-bromosiobutyrate was used at a lower concentration (1/10) than in reaction (nvt-08-007-55, i.e., 0.1094 g, 9.525×10⁻⁵ mol). The amount of catalyst added to the reaction was the same as in the above reactions but the amount of AIBN was decreased three-fold. The reaction was carried out at 65° C. for 30 h. M_{n(GPC)}=101,000 g mol⁻¹, PDI=1.26, a small amount of coupling was observed in the GPC curves.

In one embodiment of the invention some arm-arm coupling can be accepted, indeed selected, as this chemically linked structure mimics the self-assembled physical network we envision the stars to adopt when acting as efficient rheology modifies.

### Transformation of Active Chain Ends to Form a Segmented Arm.

The halogen chain ends on each PHEA arm of the star polymers can be replaced in an atom transfer coupling reaction in the presence of a large excess of a radical source. This disclosed procedure increases the fraction of cross coupling between the high molecular weight slow diffusing macro-radicals and low molecular weight radicals, in the presence of small amount of a Cu(II) complex. The radical source provides radicals that both reduce the stable higher oxidation state transition metal complex (similar to ICAR) and couple with the radicals generated via activation of the dormant polymer chain ends on each arm of the star by the generated Cu(I) activator (Scheme 4). The reaction can be used for a variety of modifications, including removal of halogen atoms from the polymer chain ends when halogen is undesirable, but is employed herein to tether oleophobic segment(s) to the termini of the arms.

For the preparation of an exemplary thickening agent the hexafunctional PHEA star copolymers, with arms of two different molecular weights, were coupled with the radicals generated in the decomposition of lauryl peroxide (Scheme 4) thereby providing a star copolymer with a PHEA core and a peripheral shell comprising tethered saturated C₁₁ alkyl chains.

### Example 5

### Synthesis of a Thickening Agent from a Six-Arm Star Polymer with polyHEA Arms and Hydrocarbon Chain Ends

### Example 5a: (nvt-08-007-58)

3.0 g of six-arm star polyHEA (apparent Mₙ (from GPC)=56,410 g mol⁻¹. One must recognize that the selection of the GPC standard and the topology of the star macromonomer make this result inaccurate nevertheless it is included to provide a relative measurement for further examples. 5 g of lauryl peroxide, 0.0212 g of CuBr₂, and 0.0276 g of TPMA were mixed with 15 mL of DMF. The heterogeneous mixture was purged for 1 h with nitrogen and was then heated to 65° C. for 16 h. The polymer was precipitated in ether and washed well with the solvent. It had very different solubility compared to the starting polyHEA. It dissolved in shampoos but not in water, whereas the behavior of polyHEA is the opposite.

### Example 5b: (nvt-08-007-59)

In another reaction, 3.0 g of six-arm star polyHEA, apparent Mₙ (from GPC)=101,000 g mol⁻¹. 5 g of lauroyl peroxide, 0.0212 g of CuBr₂, and 0.0276 g of TPMA were mixed with 15 mL of DMF. The heterogeneous mixture was purged for 2 h with nitrogen and was then heated to 70° C. for 20 h. The polymer was again precipitated in ether and washed well with ether.

### Evaluation of Star Macromolecules as Thickening Agents

Cosmetic and personnel care preparations prepared with star macromolecules of the present invention may contain the star molecules in quantities of 0.01 to 5% by weight, preferably in quantities of 0.05 to 3% by weight and more particularly in quantities of 0.1 to 2% by weight, based on the formulation as a whole.

Depending on the composition and the nature of the cosmetic preparation, the viscosity of the formulation can be adjusted to an exact value through the choice of the star molecule the composition and molecular weigh of each segment of the arms of the star. Depending on the thickened formulation, viscosities in the range from 100 to 1,000,000 mPas, preferably in the range from 1,000 to 50,000 mPas and more particularly in the range from 4,000 to 35,000 mPas as measured by a Brookfield RVT viscosimeter, 10 r.p.m., spindle 4, room temperature. Final viscosity can be adjusted by modifying the composition and molecular weight of each arm in the star in addition to the number of arms in the star.

### Example 6

### Measurement of Thickening Properties

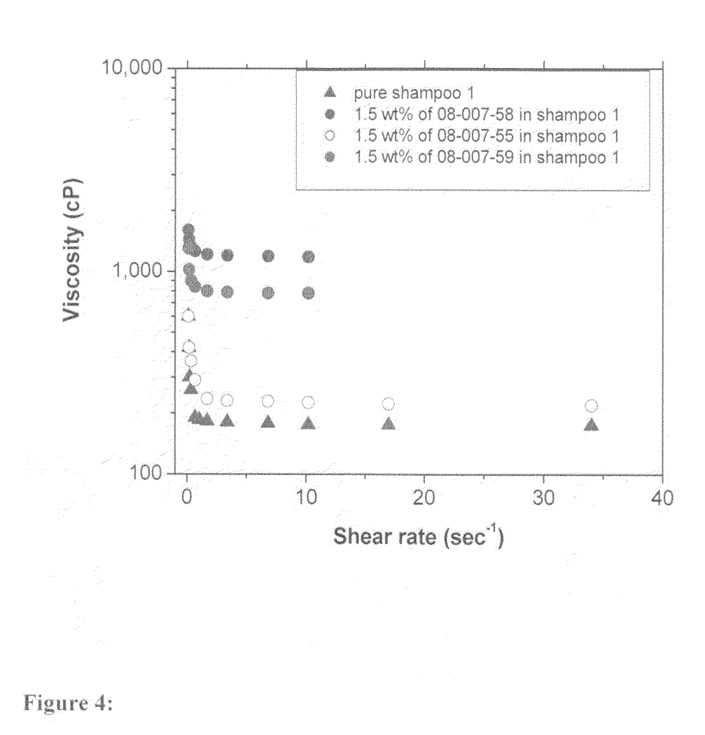

Viscosity measurement was conducted using a Brookfield LVDV-E, Spindle #31 (#34 or #25), T=25° C. The shampoo was a sulfate free shampoo #1 (US-00760-195) provided by Cognis. FIG. 4 shows that addition of 1.5 wt % of the six armed star molecule significantly changed the viscosity of the formed solution. Viscosity measurement was conducted using a Brookfield LVDV-E, Spindle #31 (#34 or #25) and a temperature=25° C. The sulfate free shampoo, #1 (US-00760-195) was provided by Cognis. The figure shows that the presence of a hydrophobic “shell” on the star modifies the viscosity of the formulation.

The present invention provides a general method for the synthesis of star polymers with pre-determinable molecular weight and narrow molecular weight distribution and pre-selected site specific functionality.

A further embodiment of the present invention provides a method for the synthesis of multi-arm star polymers where the core of the star polymers contains additional functionality.

A further embodiment of the present invention provides a method for the synthesis of multi-arm star polymers where the periphery of the star polymers contains additional functionality.

In an additional embodiment the functionality at the periphery of the star comprises molecular recognition functionality wherein the dominating non-covalent bonds responsible for the molecular recognition comprise hydrophobic segments that interact with added agents.

An embodiment of the present invention is a general method for the synthesis of star polymers comprising block copolymer arms with high molecular weight and narrow molecular weight distribution wherein the control agent employed in synthesis of the first formed star has been removed.

### REFERENCES

### US Patents

U.S. Pat. No. 5,763,548 (Co)polymers and a novel polymerization process based on atom (or group) transfer radical polymerization.
U.S. Pat. No. 5,789,487 Preparation of novel homo- and copolymers using atom transfer radical polymerization.
U.S. Pat. No. 5,807,937 Preparation of novel homo- and copolymers using atom transfer radical polymerization.
U.S. Pat. No. 5,945,491 Processes based on atom (or group) transfer radical polymerization and novel (co) polymers having useful structures and properties
U.S. Pat. No. 5,986,015 Method of making graft polymers
U.S. Pat. No. 6,111,022 Preparation of novel homo- and copolymers using atom transfer radical polymerization
U.S. Pat. No. 6,121,371 Application of atom transfer radical polymerization to water-borne polymerization systems
U.S. Pat. No. 6,124,411 Preparation of novel Homo- and copolymers using atom transfer radical polymerization
U.S. Pat. No. 6,162,882 Preparation of novel homo- and copolymers using atom transfer radical polymerization
U.S. Pat. No. 6,365,672 Polysiloxane block copolymers in topical cosmetic and personal care compositions
U.S. Pat. No. 6,407,187 (Co)polymers and a novel polymerization process based on atom (or group) transfer radical polymerization
U.S. Pat. No. 6,476,124 Composition comprising polymers having a star structure, the polymers, and their use
U.S. Pat. No. 6,512,060 Atom or group transfer radical polymerization
U.S. Pat. No. 6,538,091 Atom or group transfer radical polymerization
U.S. Pat. No. 6,552,146 Composition comprising polymers having a star structure, the polymers, and their use
U.S. Pat. No. 6,541,580 Atom or group transfer radical polymerization in the presence of transition metals
U.S. Pat. No. 6,627,314 Preparation of nanocomposite structures by controlled polymerization
U.S. Pat. No. 6,663,855 Cosmetic and personal care compositions
U.S. Pat. No. 6,692,733 Composition comprising polymers having a star structure, the polymers, and their use
U.S. Pat. No. 6,723,789 Composition comprising polymers having a star structure, the polymers, and their use
U.S. Pat. No. 6,737,071 Composition comprising polymers having a star structure, the polymers, and their use
U.S. Pat. No. 6,759,491 Simultaneous reverse and normal initiation of ATRP
U.S. Pat. No. 6,946,525 Composition comprising polymers having a star structure, the polymers, and their use
U.S. Pat. No. 7,318,929 Cosmetic preparations
U.S. Pat. No. 7,332,550 Stabilization of transition metal complexes for catalysis in diverse environments

### US Application

Ser. No. 10/887,029 Antimicrobial surfaces prepared using atom transfer radical polymerization
Ser. No. 11/990,836 Polymerization Process with catalyst reactivation

### Published Applications

WO 96/030421 Atom- or group-transfer radical polymerization and polymers produced by the process
WO 97/018247 Improved processes based on atom (or group) transfer radical polymerization and novel (co)polymers having useful structures and properties
WO 98/040415 Improvements in atom (or group) transfer radical polymerization
WO 05087818 Preparation of tetrazole polymers with controlled functionality for pharmaceutical use
WO 05/087819 Atom transfer radical polymerization in the presence of a reducing agent
WO 07/025,310 POLYMERIZATION PROCESS WITH CATALYST REACTIVATION

### Literature

Zilliox, J. G., P. Rempp, et al.; Journal of Polymer Science, Polymer Symposia (1968) No. 22 (Pt. 1): 145-56.
J. Falbe (ed.), “Katalysatoren, Tenside and Mineraloladditive (Catalysts, Surfactants and Mineral Oil Additives)”, Thieme Verlag, Stuttgart, 1978, pages 123-217.
J. Falbe (ed.), “Surfactants in Consumer Products”, Springer Verlag, Berlin, 1987, pages 54 to 124.
Kanaoka, S., N. Hayase, et al.: Polymer Bulletin (Berlin) 2000, 44(5-6): 485-492.
Kaneyoshi, H.; Inoue, Y.; Matyjaszewski, K. Macromolecules 2005, 38, 5425-5435.
Matyjaszewski, K.; et al.; Proceedings of the National Academy of Sciences of the United States of America 2006, 103, 15309-15314.
Shibata, T., S. Kanaoka, et al.; J. Am. Chem. Soc; 2006, 128(23): 7497-7504.
Tang, W.; et. al; J. Am. Chem. Soc. 2008, 130, 10702-10713.
Lin, C. Y.; Coote, M. L.; Gennaro, A.; Matyjaszewski, K. J. Am. Chem. Soc. 2008, 130, 12762-12774.

1. A star macromolecule comprising three or more arms wherein each arm comprises a segmented copolymer wherein the “star” structure comprises molecules of the following formulae,
either:
C-[(S1)ₚ₁-(S2)ₚ₂]ₙ; or
{C}_{L}-(S1)ₚ₁-(S2)ₚ₂]_{n′} in which:
C represents a polyfunctional star centre or core, with a functionality of “n”, n being an integer greater than or equal to 3, preferably between 3 and 25, and {C}_{L}- represents the situation when several, two or more, (L) polyfunctional C centers are linked together to provide a central core with an increased number of arms;
-[(S1)ₚ₁-(S2)ₚ₂] represents a polymeric chain, also known as an “arm” of the star, composed of different polymerized monomers wherein segment S1, the inner tethered segment of each arm comprises one or more comonomer units and S2 comprises the second, outer oligo/polymer segment, on each branch,
Each -[(S1)ₚ₁-(S2)ₚ₂] arm being identical or different compositions but each being covalently grafted from the centre C;
n and n′ being the initial number of initiating functionalities on the polyfunctional center or core which is greater than or equal to 3, preferably of between 3 and 25;
p1 being the degree of polymerization of monomers comprising segment S1 which is greater than or equal to 2, preferably of between 10 and 20,000;
p2 being the degree of polymerization of monomers comprising segment S2 which is greater than or equal to 1, preferably of between 1 and 1,000.

2. A star macromolecule wherein the inner shell of the tethered arms, S1 in claim 1, comprise a branching agent thereby increasing the number of arms present on the periphery of the inner shell of the star beyond the number of initiator moieties initially present on the added multifunctional initiator.

3. The star macromolecules of claim 1 or 2 comprising inner shell segments that are stable in the presence of salts and over a range of pH.

4. The star macromolecule of claim 3 wherein the inner shell or core of the star comprises a water soluble non-ionizable monomer.

5. The star macromolecule of claim 4 wherein the inner segments of the arms comprise non-ionizable hydrophilic segments selected to make the star macromolecules compatible with solutions further comprising dissolved/dispersed salts thereby providing rheology modifiers that are stable over a broad range of pH.

6. The star macromolecule of claim 5 wherein the rheology modifiers are stable over a range of pH from 1 to 14.

7. The star macromolecules of claim 1 wherein the core of the star comprise degradable functionality.

8. The star macromolecule of claim 1 wherein the terminal segments on one or more of the tethered arms comprise a polymer composition that is compatible with added agents in the targeted application to provide an additional degree of self assembly thereby providing an additional level of control over thickening properties.

9. The star macromolecule of claim 8 wherein the terminal segment comprises an aliphatic chain end compatible with hydrophobic functional groups present in added commercially available surfactants including anionic, nonionic, cationic, zwiterionic and amphoteric surfactants.

10. The star macromolecule of claim 8 wherein the terminal segment comprises monomer units with oleophobic substituents.

11. A process for the preparation of star macromolecules of claim 1 with arms comprising two or more segments wherein the inner shell of the star is formed by conducting a controlled radical polymerization of monomers providing a first hydrophilic segment from a multifunctional initiator then reactivating the polymer chain ends in the presence of radicals comprising a desired functional group/oligo-polymer segment of differing phylicity thereby removing the control agent from the first tethered chain ends and trapping an additional functional group/oligo-polymer segment in an outer shell of the star macromolecule.

12. The star macromolecule of claim 11 wherein the additional peripheral functional group/oligo-polymer segment comprises an aliphatic chain.

13. The star macromolecule of claim 11 wherein the first formed polymer segments in each star arm comprise a non-ionizable hydrophilic polymer segment.

14. The star macromolecule of claim 11 wherein the first formed polymer segments comprise monomers selected from 2-hydroxylethyl (meth)acrylates, hydroxypropyl (meth)acrylates, glycidyl (meth)acrylates, PEO-oligo (meth)acrylates, (meth)acrylamides, allyl alcohol and vinyl pyrrolidone.

15. The star macromolecule of claim 11 wherein the hydrophobic arm segments interact with hydrophobic segments in adjacent star molecules to self-assemble into a three dimensional array when dispersed in aqueous media.

16. The star macromolecule of claim 15 wherein the hydrophobic arm segments interact with surfactant molecules present in the media in addition to hydrophobic segments in adjacent star molecules to self-assemble into a three dimensional array when dispersed in aqueous media.

17. A process for the preparation of star macromolecules of claim 1 with arms comprising two or more segments wherein the inner shell or core of the star is formed by conducting a controlled radical polymerization of monomers providing a first hydrophobic segment from a multifunctional initiator then reactivating the polymer chain ends in the presence of radicals comprising a desired functional group/oligo-polymer segment of differing phylicity thereby removing the control agent from the first chain end and trapping an additional functional group/oligo-polymer segment.

18. The star macromolecules of claim 17 with segmented arms wherein the radically transferable atom or group on the periphery of the first formed multi-armed star is converted into an oleophylic segment in an atom transfer radical trapping reaction.

19. The process of claim 11 or 17 wherein the controlled polymerization process is an atom transfer radical polymerization process.

20. The star macromolecules of claims 11 and 17 with segmented arms wherein there are no remaining transfer/capping agent at the chain ends.

21. The star macromolecules of claims 11 and 17 with segmented arms wherein the terminal polymer segments comprise chain ends compatible with functional groups present in added commercially available surfactants including anionic, nonionic, cationic, zwiterionic and amphoteric surfactants.

22. The star macromolecules of claims 11 and 17 with segmented arms wherein the multifunctional initiator forming the core of the star, C in formula (1) is prepared using an AB* inimer.

23. The star macromolecules of claims 11 and 17 with segmented arms wherein the inner shell of the star macromolecule comprise a fraction of AB* monomer units equal to the number of initial initiating sites can be added to form a branch in the growing polymer chain thereby increasing the number of active chain ends for conversion to oleophilic units thereby increasing the number of outer arm segments without increasing congestion at the core of the star

24. The star macromolecules of claims 11 and 17 with segmented arms wherein multifunctional core of the star can comprise natural biodegradable polysaccharides.

25. A cosmetic or personal care composition comprising a star macromolecule of claim 11 with three or more arms where each arm comprises an inner hydrophilic segment and an outer hydrophobic segment that functions as a rheology modifier.

26. A cosmetic or personal care composition comprising a star macromolecule of claim 17, with three or more arms where each arm comprises an inner hydrophobic segment and an outer hydrophylic segment that functions as a rheology modifier.

27. The cosmetic or personal care composition of claims 25 and 26 that function as a rheology modifier in the presence of added salts and over a broad range of pH.

28. The cosmetic or personal care composition of claims 25 and 26 wherein the concentration of the star macromolecule is less than 5%, preferably less than 2%.
